(19) United States
(12) Patent Application Publication
(10) Pub. No.: US 2005/0106128 A1
(43) Pub. Date: May 19, 2005

(54) Novel methods of treatment and deliver modes

(76) Inventors: Robert Bartlett Elliott, Auckland (NZ); Stephen John Martin Skinner, Auckland (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/494,820

(22) PCT Filed: Nov. 1, 2002
(86) PCT No.: PCT/NZ02/00235

(30) Foreign Application Priority Data
Nov. 7, 2001 (NZ) 515310

(51) Int. Cl. A61L 9/04; A61K 9/14

(52) U.S. Cl. 424/93.7; 424/46

(57) ABSTRACT

The invention relates to a novel method of administration, and device employed in the method, for administering a treatment species to the lungs of a recipient patient. The method involves introducing the device of the invention to the venous system of the patient, the size of the treatment species being such that, upon introduction to the venous system of the patient, the device will impact in a region of a lung capillary of the patient. The treatment species gains access to the lung and/or lung epithelia due to proteases associated with the treatments species. The application of the method to the treatment of cystic fibrosis is also claimed.

### FIELD OF THE INVENTION

The invention relates to novel delivery modes for the treatment of mammals. Particularly but not exclusively the invention relates to novel methods of treatment in situ in organs of mammals, particularly the lungs. The invention also relates to treatment regimes applicable to treatment of cystic fibrosis.

### BACKGROUND

### Lung Illnesses and Conditions

Illnesses and conditions of the lungs are varied and widespread. They vary from asthma, cancer, emphysema, to congenital cilia problems and cystic fibrosis for example. Such illnesses and conditions have one important characteristic in common—the difficulty of treatment as a result of in accessibility.

1. Cystic Fibrosis

Cystic fibrosis (CF) is a fatal genetic disease primarily affecting Caucasians, although cases have been reported from other ethnic groups. It is chiefly a disease of electrolyte transport being an inability of the membranes lining the airways to produce sufficiently hydrated secretions. This causes blockages of the bronchial airways and development of recurrent infections in the lungs. Similar changes occur in other organs of the body, but these are not so life threatening.

People with Cystic Fibrosis suffer from chronic lung problems and digestive disorders. The lungs of people with Cystic Fibrosis become covered with a sticky mucus which is hard to remove and promotes infection by bacteria. Many people with CF require frequent hospitalizations and continuous use of antibiotics, enzyme supplements, and other medications.

The underlying cause is a defective electrolyte ‘pump’ in the cells lining the airways, which in return is due to inherited gene abnormality. Humans have a gene encoded in their DNA which manufactures a special protein called CFTR (Cystic Fibrosis Transmembrane Conductance Regulator). The CFTR protein is a member of a family of ATP-binding proteins that act as unidirectional solute pumps. This protein controls the flow of chloride ions across the cell membrane.

Each gene is made up of two alleles; a single correctly encoded allele is adequate for normal CFTR production. Thus it is only when a person has two defective CFTR alleles that they actually have Cystic Fibrosis. Those with a single defective allele are called carriers, and those with two defective alleles have Cystic Fibrosis.

The gene(s) causing the defect are known and how they cause the defective electrolyte ‘pump’ is also known. Attempts to replace the defective gene in a person with the disease by genetic engineering appear to have failed, despite much time, effort, expertise and money being expended. This approach may yet be successful in the long term.

### Treatment of Cystic Fibrosis

Since the defective CF gene was discovered in 1989, the pace of CF research has greatly accelerated. In 1990, scientists successfully made copies of the normal gene, and added them to CF cells in laboratory dishes, which corrected the defective cells. The next major step was achieved in early 1993 when the first experimental gene therapy treatment was given to a patient with CF. Researchers modified a common cold virus to act as a delivery vehicle—carrying the genes to the CF cells in the airways. Several studies are underway to test new gene delivery methods, such as fat capsules (liposomes) and synthetic vectors.

The first new drug therapy developed exclusively for CF in 30 years was approved by the Food and Drug Administration (FDA) in 1993. In clinical trials, this mucus-thinning drug called Pulmozyme®, reduced the number of respiratory infections and improved lung function. In 1995, a four-year study showed that the drug, ibuprofen, reduced the rate of lung inflammation in children with CF—under controlled conditions, and in high doses.

In late 1997, the FDA approved the drug TOBI™ (tobramycin solution for inhalation). In clinical trials, this reformulated version of the common antibiotic improved lung function in people with CF and reduced the number of hospital stays. The benefits of TOBI are that it can be delivered in a more concentrated dose directly to the site of CF lung infections more efficiently, and that it is preservative-free. The development of TOBI should lead to a long line of other aerosolized antibiotics for people with CF.

### Cell/Organ Transplantation to Treat CF

Whole lung transplantation has been carried out successfully with cure of the lung disease, but this is technically difficult, expensive, has limited success and relies on availability of organ donors.

The transplanted lungs came from individuals who do not have CF. These “new” lungs are initially disease free, however, CF does remain in the sinuses, pancreas, intestines, sweat glands, and reproductive tract after the lung transplant.

Each individual has an immune system that protects against foreign material, including microbes. Transplanted organs are foreign to the recipient and the immune system reacts against them in a process called “rejection.” Immunosuppressive drugs are given daily for the life of each transplant recipient to reduce the immune response and protect the transplanted organs from rejection. Immunosuppressive drugs may increase one's susceptibility to some infections, and cause side effects such as diabetes, decreased kidney function, and osteoporosis (thinning of the bones). The doses of such drugs are adjusted to maintain adequate immunosuppression and minimize these side effects.

An alternative approach could be replacement of at least some of the defective cells by transplantation. It is known that even a very small increase in the electrolyte ‘pumping’ will reverse the disease i.e. only a small percentage of cells need to be replaced in the airway lining.

### OBJECT OF THE INVENTION

It is an object of the invention to provide a novel means of treating mammals suffering from diseases or conditions which manifest themselves in the lungs of the mammal; and it is a further or alternative object of the invention to provide a novel or alternative treatment for the condition of cystic fibrosis

### STATEMENTS OF THE INVENTION

According to a first aspect of the invention there is provided a biological device capable upon impaction in a lung capillary of a patient suffering from cystic fibrosis of treating the patient comprising or including:
i) one or more cells exhibiting substantially normal CFTR production;
ii) one or more coatings of a suitable biocompatible material around the one of more cells;
iii) one or more proteases associated with the one or more coatings;

wherein the size of the biological device is such that, upon introduction to the venous system of the patient the device will impact substantially in the region of a lung capillary of the patient,

and wherein, upon impaction, by disruption of the outer surface of the device or otherwise, the one or more proteases are able to act upon a wall of the lung capillary, thereby allowing access (directly or indirectly) of the one or more cells to the lung epithelia

Preferably the diameter of the biological device is substantially within the range 20-80 micrometers.

Preferably the one or more proteases are distributed substantially uniformly in the one or more coatings, whether it is uniformly through all of the one or more coatings, or uniformly within one or more of the one or more coatings. Alternatively the one or more proteases are distributed throughout the one or more coatings in clusters.

Preferably the one or more proteases have the characteristics of proteases secreted by the Ascaris roundworm. Preferably the one or more proteases are neutral proteinases. More preferably the one or more proteases may be collagenases or proteo glycanases. Preferably the cells may include human lung cells and/or porcine lung cells and/or human lung stem cells.

Preferably the one or more coatings around the cells provide(s) a protective coating to the cells and is//are permeable to nutrients. Preferably nutrients include water, salts and glucose.

Preferably the one or more coatings is/are or include alginate.

Preferably the one or more coatings comprise the following:
i) an inner layer of alginate,
ii) polyornithine,
iii) an outer layer of alginate.

Preferably the proteases are in clusters, contained within a microcapsule, and are held in or on the outer layer of alginate. Preferably the microcapsule may be of a suitable water absorbing material, preferably gelatin.

Preferably the one or more cells have been obtained according to a process of isolation from a donor.

Preferably the process of isolation includes exposure of the one or more cells to a liquid medium containing one or more of:
Nicotinamide,
Liberase/Collagenase,
Lignocaine.

Preferably the biological devices is contained within or supported by a pharmaceutically acceptable intravenous carrier.

According to a second aspect of the invention there is provided a method of preparing a biological device capable, upon impaction in a lung capillary of a patient suffering from cystic fibrosis, of treating the patient comprising or including the steps of:
i) isolation of one or more cells exhibiting substantially normal CFTR production;
ii) a step of coating the one or more cells with one or more coatings of a suitable biocompatible material;
iii) attaching or otherwise associating one or more proteases with the one or more coatings;
   wherein the size of the biological device is such that, upon introduction to the venous system of the patient the device will impact substantially in the region of a lung capillary of the patient.

Preferably the diameter of the biological device is substantially in the range 20-80 micrometers.

Preferably the one or more cells are human lung cells and/or porcine lung cells and/or human lung stem cells.

Preferably nicotinamide and/or Lignocaine may be introduced to the one or more cells prior to coating, or at any one or more stages of the procedure.

Preferably the coating step includes the following substeps:
(a) encapsulation or encasement of the one or more cells in a suitable biocompatible material,
(b) coating the encapsulated one or more cells with a positively charged material,
(c) providing an outer coat of a suitable biocompatible material.

Preferably the biocompatible material employed in (a) and (c) is a suitable alginate.

Preferably the alginate is in ultra pure form.

Preferably the encapsulation provides a surround which prevents, once implanted, direct tissue contact with the one or more cells.

Preferably each encapsulation involves presenting the one or more cells and a suitable alginate solution into a source of compatible cations thereby to entrap the one or more cells in a cation—alginate gel.

Preferably said cation alginate gel is calcium-alginate gel.

Preferably said alginate used in the solution is sodium alginate, and the islet and sodium alginate solution is presented as a droplet into a bath of suitable cations.

Preferably the second layer of the capsule will be of a positively charged polymer material preferably poly-L-ornithine.

Preferably the coatings comprise the following:
i) an inner layer of alginate,
ii) polyornithine,
iii) an outer layer of alginate.

Preferably step iii) includes substantially uniform distribution of the one or more proteases in the one or more coatings (by mixing with the outer coating before application for example). Alternatively step iii) includes distributing the one or more proteases throughout the outer coating in clusters (by mixing the clusters with the outer coating before application for example).

Preferably the one or more proteases have the characteristics of proteases secreted by migrating parasitic nematodes, more preferably Ascaris suum, or stercoralis. Preferably the one or more proteases are neutral proteinases. More preferably the one or more proteases may be collagenases or proteoglycanases.

According to a further aspect of the invention there is provided an intravenous preparation for administration to a patient suffering from cystic fibrosis comprising or including:
(a) a biological device as previously described, and
(b) a pharmaceutically acceptable intravenous carrier.

Preferably the intravenous preparation may be stored at a range of temperatures not less that 2° C. and not exceeding 30° C. without destabilisation and/or decomposition.

According to a further aspect of the invention there is provided a method of treating a patient suffering from cystic fibrosis comprising or including the step of:
Intravenous administration to the patient of an intravenous preparation comprising or including:
   (a) a pharmaceutically acceptable intravenous carrier, and
   (b) a biological device,
      wherein the biological device is capable, upon impaction in a lung capillary of a patient suffering from cystic fibrosis, of treating the patient.

Preferably the biological device comprises or includes:
i) one or more cells exhibiting substantially normal CFTR production;
ii) one or more coatings of a suitable biocompatible material around the one of more cells;
iii) one or more proteases associated with the one or more coatings;
and wherein the size of the biological device is such that, upon introduction to the venous system of the patient the device will impact substantially in the region of a lung capillary of the patient,
and wherein, upon impaction, by disruption of the outer surface of the device or otherwise, the one or more proteases are able to act upon a wall of the lung capillary, thereby allowing access (directly or indirectly) of the one or more cells to the lung epithelia.

Preferably the diameter of the biological device is substantially within the range 20-80 micrometers.

Preferably the one or more proteases are distributed substantially uniformly in the one or more coatings, whether it is uniformly through all of the one or more coatings, or uniformly within one or more of the one or more coatings. Alternatively the one or more proteases are distributed throughout the one or more coatings in clusters.

Preferably the one or more proteases have the characteristics of proteases secreted by migrating parasitic nematodes, more preferably Ascaris suum, or stercoralis. Preferably the one or more proteases are neutral proteinases. More preferably the one or more proteases may be collagenases or proteo glycanases.

Preferably the cells may include human lung cells and/or porcine lung cells and/or human lung stem cells.

Preferably the one or more coatings around the cells provide a protective coating to the cells and are permeable to nutrients. Preferably nutrients include water, salts, glucose and amino acids.

Preferably the one or more coatings are or include alginate.

Preferably the coatings comprise the following:
i) an inner layer of alginate,
ii) polyornithine,
iii) an outer layer of alginate.

Preferably the proteases are in clusters, contained within a microcapsule, and are held in or on the outer layer of alginate. Preferably the microcapsule may be of a suitable water absorbing material, preferably gelatin.

Preferably the one or more cells have been obtained according to a process of isolation from a donor.

Preferably the process of isolation includes exposure of the one or more cells to a medium containing one or more of:
Nicotinamide,
Liberase/Collagenase,
Lignocaine.

Preferably the patient prior to and/or during and/or after administration of the intravenous preparation is treated with an oral dose of nicotinamide.

According to a further aspect of the invention there is provided a biological device capable upon impaction in a lung capillary of a patient suffering from a condition or illness (“the condition”) of the lung or lung region, of treating the condition, comprising or including:
i) one or more treatment species capable of treating the condition,
ii) One or more proteases associated with the one ore more treatment species,
wherein the size of the treatment species is such that, upon introduction to the venous system of the patient, the device will impact substantially in a region of a lung capillary of the patient,
and wherein, upon impaction by disruption of the outer surface of the device or otherwise, the one or more proteases are able to act upon a wall of the lung capillary, thereby allowing access (directly or indirectly) of the one or more treatment species to the lung epithelia.

Preferably the device includes one ore more external coatings of a biocompatible material around the one or more treatment species, the one or more proteases being distributed within the one ore more external coatings, and the diameter of the device is substantially within the range 20-80 micrometers.

Preferably the one or more proteases have the characteristics of proteases secreted by the Ascaris roundworm, and are neutral proteinases.

According to a further aspect of the invention there is provided a method of preparing a biological device capable, upon impaction in a lung capillary of a patient suffering from a condition or illness (“the condition”) of the lung or lung region of treating the condition comprising or including the steps of:
i) isolation of one or more treatment species capable of treating the condition;
ii) a step of coating the one or more cells with one or more coatings of a suitable biocompatible material;
iii) attaching or otherwise associating one or more proteases with the one or more coatings;
wherein the size of the biological device is such that, upon introduction to the venous system of the patient the device will impact substantially in the region of a lung capillary of the patient.

Preferably the diameter of the biological device is substantially in the range 20-80 micrometers.

According to a further aspect of the invention there is provided an intravenous preparation for administration to a patient suffering from a condition or illness of the lung or lung region comprising or including:
(a) a biological device as described above, and
(b) a pharmaceutically acceptable intravenous carrier.

According to a further aspect of the invention there is provided a method of treating a patient suffering from a condition or illness (“the condition”) of the lung or lung region comprising or including the step of:
Intravenous administration to the patient of an intravenous preparation comprising or including:
   (a) a pharmaceutically acceptable intravenous carrier, and
   (b) a biological device,
wherein the biological device is capable, upon impaction in a lung capillary of a patient suffering from the condition, of treating the patient.

Preferably the diameter of the biological device is substantially within the range 20-80 micrometers.

Preferably the biological device is as described above.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### Definitions

As used herein these terms have the following meanings:
CFTR—the Cystic Fibrosis Transmembrane Conductance Regulator protein having a functioning chloride pump.
Chloride Pump—functions in the epithelial cells, taking chloride from the airway fluids and moving to the serosal side of the epithelial layer.
Cells exhibiting normal CFTR production—cells which substantially have a functioning chloride pump secreting producing water into the airway fluids.
Impact/Impaction—describes the process where the biological device, due predominantly to its size, is prevented from passing further through the capillary and is effectively jammed
Diameter of the Device—describes a measurement from side to side of the device; it downs not necessarily imply that the device is spherical although it may be spherical.

Other objects of the invention may become apparent from the following description which is given by way of example only.

Other aspects of the invention may become apparent from the following description which is given by way of example only and with reference to the accompanying drawings.

### BRIEF DESCRIPTION OF THE DRAWINGS

The invention will now be described by way of example only and with reference to the drawings in which:

FIG. 1: illustrates a lung cell cluster of porcine lung epithelia

FIG. 2: illustrates a number of the clusters of FIG. 1,

FIG. 3: illustrates the preparation of a biological device in accordance with the invention,

FIG. 4: illustrates the process of impaction in a lung capillary of a device of the invention.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### DETAILED DESCRIPTION OF THE INVENTION

1. Background

The novel administration and treatment means of the invention results from knowledge of a parasitic nematode, the Ascaris roundworm life cycle in human beings, as detailed below.

2. Intestinal Roundworms

Ascaris lumbricoides is one of the largest and most common parasites found in humans. The adult females of this species can measure up to 18 inches long (males are generally shorter), and it is estimated that 25% of the world's population is infected with this nematode. The adult worms live in the small intestine and eggs are passed in the faeces. A single female can produce up to 200,000 eggs each day.

About two weeks after passage in the faeces the eggs contain an infective larval or juvenile stage, and humans are infected when they ingest such infective eggs. The eggs hatch in the small intestine, the juvenile penetrates the small intestine and enters the circulatory system, and quickly the juvenile worm makes its way to the capillaries of the lungs.

In the lung capillaries the juvenile worm secrets proteolytic enzymes from its mouth. These enzymes act upon the cells of the capillary wall. The wall ultimately breaks down and the worm is able to move across the blood-air barrier into the lung.

The juvenile worm then migrates up the air passages into the pharynx where it is swallowed, and once in the small intestine the juvenile grows into an adult worm.

Examples of specific worm proteases include the Strongyloides stercoralis—the larvae of this nematode parasite can move through tissue at speeds of up to 10 cm per hour. This nematode larvae secrete a potent histolytic metalloprotease to facilitate the rapid migration. This protease has elastase activity and catalyses the degradation of a model of dermal extracellular matrix.

Ascaris suum, in the tissue-invasive infective and lung stage larvae release proteinases. Specifically, this activity contained multiple proteolytic enzyme activities, particularly chymotryptic, tryptic collagenolylic and elastolytic activities.

3. Basis for the Invention

The novel administration means of the invention employs the method of blood-air barrier movement exhibited by the worm. The active agent, which in the examples considered here, is useful in treatment of cystic fibrosis, is brought into the vicinity of the lung capillary and, with the excretion or otherwise application of proteases, is able to cross the boundary into the lung.

It will be appreciated by one skilled in the art that although this discussion is primarily concerned with cystic fibrosis the novel administration method of the invention may well be used to treat other lung conditions as it allows a cell or a treatment species access to the lung. In the case of cystic fibrosis the “treatment species” is one or more cells having normal CFTR production. In other applications the treatment species may be other drugs (anticancer, asthma drugs etc) or other chemical or biological bio-actives for which will have some effect in the lung. An essential process in the invention is access of the treatment species into the lung via access to the lung epithelia. The result could be incorporation of the treatment species (or a derivative) into the epithelial layer as is the case with CFTR functioning cells. Alternatively access to the lung epithelia of the treatment species could result in access through the epithelia by disruption or otherwise, of the treatment species into the lung itself.

4. Agents Active in Cystic Fibrosis Treatment

The “active agent” contemplated here is cellular material from a suitable mammal donor. More specifically it may take one of (but is not restricted to) three forms:
i) human cells in which the CFTR protein is present (in other words, the chloride pump is functioning);
ii) porcine cells in which the CFTR protein is present;
iii) human stem cells.
   5. Biological Delivery Device

The cells are administered in the form of a biological delivery device. This is more specifically encapsulated cells, or encapsulated cell clusters.

The following outlines our preferred methodology for creation of the device but it will be appreciated other known variations or alternatives for this methodology may also be included without departing from the scope of the invention.

a. Cell Preparation

For cell transplantation the cells used may include (as above):
cells taken from healthy humans (not having Cystic Fibrosis)
cells taken from other suitable mammalian species (such as pigs)
cells taken from transgenic species not having the defective gene.
Human stem cells.

b. Cell Cluster Preparation

In our preferred method we prepare clusters of cells which will then be encapsulated. As the size of the overall device is crucial in the method of the invention (ultimately a device in range 20-80 micrometers is desired) then a cluster of <70 micrometers is (pre-encapsulation) required to provide this size.

We have prepared micro clusters of porcine lung epithelia according to the following method.
i. dissection of parenchyma from large vessels and airways of the donor species
ii. removal of red blood cells
iii. digestion with liberase or similar
iv. addition of nutrient media (including RPMI, nicotinamide, human serum albumin, pig serum, aproxin)
v. removal of gross clumps by filtration
vi. segmentation and resuspension in the nutrient medium
vii. culture in non-adherent culture dishes (up to one month)

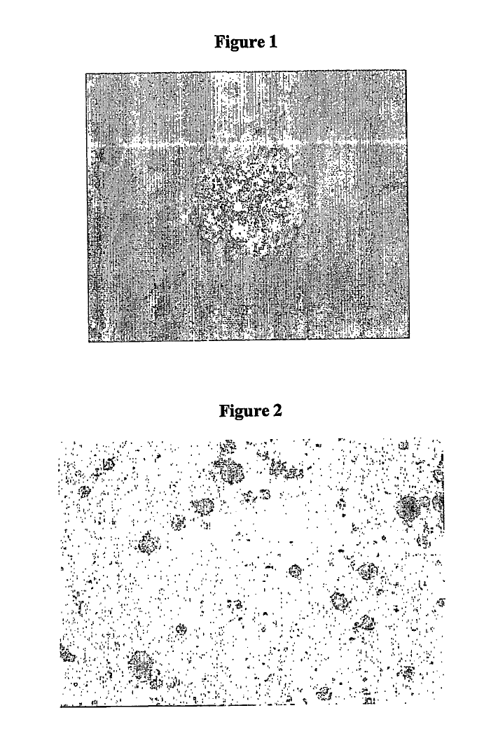

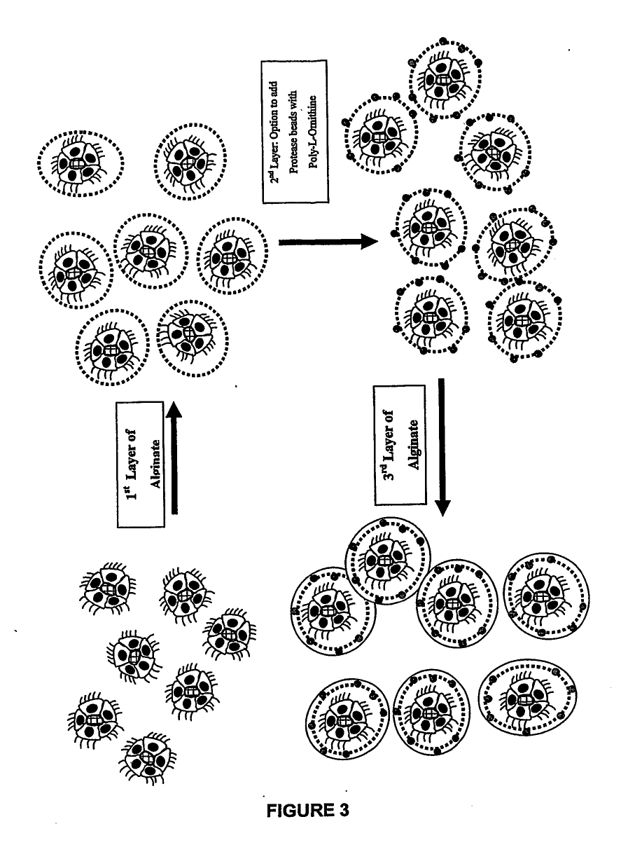

An example lung cluster prepared by such a method is shown in FIG. 1. The image is a UV/phase contrast the spheroid being some 60 micrometers in diameter. FIG. 2 illustrates a number of such clusters.

c. Encapsulation

Our cell transplant work has shown that transplanted cells even from foreign species can be protected from rejection after transplantation without the need to use severe immune suppressing drugs.

This is done by coating the cells or clusters of such cells with ‘micro-capsules’, which allow the required cell secretion out, and nutrients in but excludes the larger components of the immune system.

Smaller components can be neutralised by use of nicotinamide—a harmless vitamin derivative.

One particular encapsulation process (as an example) follows. It employs alginate as an encapsulation material but equally other in vivo similarly behaving materials may be employed.
take a population of cells (or cell clusters) to be transplanted
encapsulate or encase in an alginate coating
apply polyornithine
coat once more in alginate
The proteolytic enzyme may be inserted on top of the polyornithine before application of the second coating of alginate. Alternatively it may be mixed with the final alginate coating in the form, for example as gelatin (or other suitable) microcapsules.

This process results in encapsulated cells or cell clusters.

The outermost alginate coating will dissolve relatively quickly in blood of the patient (for example within 2 days) to expose the proteolytic enzyme.

With particular reference to FIG. 3, a form of preferred device of the invention is illustrated. In particular we have shown preparation of triple-layered encapsulation lung epithelial structures, as organotypic structures with beaded protease clusters.
(a) Porcine lung epithelia are prepared in modified cell culture media as spheroidal organotypic structures, the spheroid having an outer layer of epithelial cells a lumen filled with liquid.
(b) A first layer of alginate outside the cells is deposited by calcium gelation.
(c) The outer surface of the alginate is stabilised with a layer of poly-L-ornithine. Preferably the beaded clusters of proteases are deposited with the poly-L-ornithine layer.
(d) There is an option for a third layer of alginate to cover the protease beads in order to conserve their activity.
   6. Mode of Administration

The delivery mode takes advantage of the circulation system, in particular the venous system. The delivery device is injected into a vein and then moves through the system until it reaches the smaller diameter capillaries of the lungs.

As a result of the decreasing capillary size the device will eventually get “jammed” or impact in the vessel in the lung. The impaction may also cause compaction of the device.

The size of the device is crucial to the working of the invention. It must be large enough to impact in the capillary system within the lung but small enough not to lodge earlier in the venous system.

Lung capillaries are approximately 7-13μ diameter. This lung microvasulative has a diameter less than 100μ.

Once the device is impacted, via the structure of the outer wall of the device is destabilised and the proteases released such that they come into contact with the capillary wall.

The capillary wall will then breakdown admitting the (residue of) the device, and specifically the treatment cells. The treatment cells then, come into contact with the epithelial cells inside the lung surface. As has been known in the prior art the similar properties of the treatment cells allow merging of the treatment cells with the epithelial cells to form micro chimaeric clusters within the lung (a mixture of the two cells types).

Ultimately, the human capillary wall reorganises itself whilst the epithelial cells now include treatment cells with a healthy chloride pump activity on the lung wall.

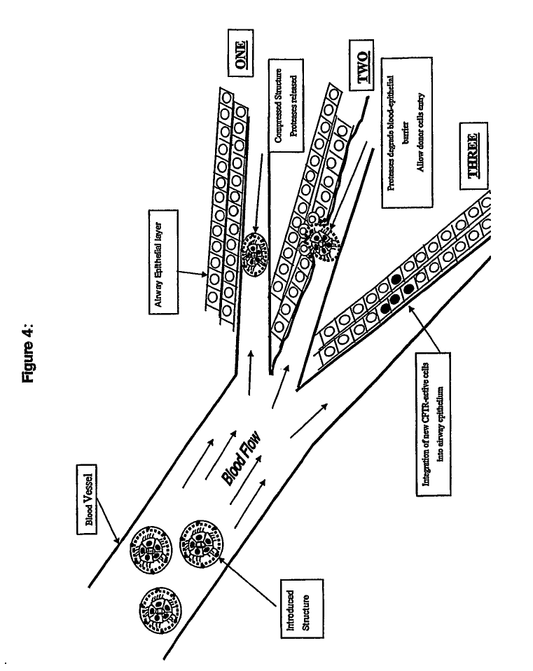

With particular reference to FIG. 4 we have illustrated an example of impaction of encapsulated lung structures through blood-air barrier and integration into the patient's airway structure.
In stage ONE the capsules are injected into a suitable vein, travel in the venous blood to the lung where the narrow capillaries prevent onward movement and the structure is impacted and compressed and the capsule structure compromised.
In stage TWO the outer surface of the capsule structure is sufficiently compromised to release the protease beads that degrade the capillary wall and the basal layer of the airway epithelium, releasing epithelial cells in a focal area.
In stage THREE the encapsulated cells are released from the capillary into the epithelial layer where they integrate as a micro-chimaeric group of cells capable of expressing CFTR and promoting chloride transport and water secretion.
   7. Outcome and Advantages

The assimilated cells should start to cause water transport into the lung linings via the chloride pumping system.

Cystic fibrosis studies have shown you only need <1% of total chloride pumping ability to significantly decrease Cystic fibrosis symptoms.

Administration is via the venous system thus the administered devices may proceed via the capillary system to all areas of the lung. This is an advantage over prior art treatment methods which generally only allow treatment in one specific area.

Where in the foregoing description reference has been made to elements or integers having known equivalents, then such equivalents are included as if they were individually set forth.

Although the invention has been described by way of example and with reference to particular embodiments, it is to be understood that modifications and/or improvements may be made without departing from the scope or spirit of the invention.

1. A biological device capable, upon impaction in a lung capillary of a patient suffering from cystic fibrosis, of treating the patient comprising or including:
i) one or more cells exhibiting substantially normal CFTR production;
ii) one or more coatings of a suitable biocompatible material around the one of more cells;
iii) one or more proteases associated with the one or more coatings; wherein the size of the biological device is such that, upon introduction to the venous system of the patient the device will impact substantially in the region of a lung capillary of the patient, and wherein, upon impaction, by disruption of the outer surface of the device or otherwise, the one or more proteases are able to act upon a wall of the lung capillary, thereby allowing access (directly or indirectly) of the one or more cells to the lung epithelia.

2. A device as claimed in claim 1 wherein the diameter of the biological device is substantially within the range 20-80 micrometers.

3. A device as claimed in claim 2 wherein the one or more proteases are distributed substantially uniformly in the one or more coatings, whether it is uniformly through all of the one or more coatings or uniformly within one or more of the one or more coatings.

4. A device as claimed in claim 2 wherein the one or more proteases are distributed throughout the one or more coatings in clusters.

5. A device as claimed in claim 3 or 4 wherein the one or more proteases have the characteristics of proteases secreted by the Ascaris roundworm.

6. A device as claimed in claim 5 wherein the one or more proteases are neutral proteinases.

7. A device as claimed in claim 6 wherein the one or more proteases are collagenases or proteo glycanases.

8. A device as claimed in claim 7 wherein the cells may include human lung cells and/or porcine lung cells and/or human lung stem cells.

9. A device as claimed in claim 8 wherein the one or more coatings around the cells provide(s) a protective coating to the cells and is/are permeable to nutrients, including one or more of water, salts and glucose.

10. A device as claimed in claim 9 wherein the one or more coatings is/are or include alginate.

11. A device as claimed in claim 10 wherein the one or more coatings comprise the following:
i) an inner layer of alginate,
ii) polyornithine,
iii) an outer layer of alginate.

12. A device as claimed in claim 11 wherein the proteases are in clusters, contained within a microcapsule, and are held in or on the outer layer of alginate.

13. A device as claimed in claim 12 wherein the microcapsule is of a suitable water absorbing material.

14. A device as claimed in claim 13 wherein the suitable water absorbing material is gelatin.

15. A device as claimed in claim 14 wherein the one or more cells have been obtained according to a process of isolation from a donor.

16. A device as claimed in claim 15 wherein the process of isolation includes exposure of the one or more cells to a liquid medium containing one or more of:
1. Nicotinamide,
2. Liberase/Collagenase,
3. Lignocaine.

17. A device as claimed in claim 16 wherein the biological device is contained within or supported by a pharmaceutically acceptable intravenous carrier.

18. A method of preparing a biological device capable, upon impaction in a lung capillary of a patient suffering from cystic fibrosis, of treating the patient comprising or including the steps of:
i) isolation of one or more cells exhibiting substantially normal CFTR production;
ii) a step of coating the one or more cells with one or more coatings of a suitable biocompatible material;
iii) attaching or otherwise associating one or more proteases with the one or more coatings;
wherein the size of the biological device is such that, upon introduction to the venous system of the patient the device will impact substantially in the region of a lung capillary of the patient.

19. A method as claimed in claim 18 wherein the diameter of the biological device is substantially in the range 20-80 micrometers.

20. A method as claimed in claim 19 wherein the one or more cells are human lung cells and/or porcine lung cells and/or human lung stem cells.

21. A method as claimed in claim 20 wherein nicotinamide and/or Lignocaine is/are introduced to the one or more cells prior to coating, or at any one or more stages of the procedure.

22. A method as claimed in claim 21 wherein the coating step includes the following substeps:
(a) encapsulation or encasement of the one or more cells in a suitable biocompatible material,
(b) coating the encapsulated one or more cells with a positively charged material,
(c) providing an outer coat of a suitable biocompatible material.

23. A method as claimed in claim 22 wherein the biocompatible material employed in (a) and (c) is a suitable alginate.

24. A method as claimed in claim 23 wherein the encapsulation provides a surround which prevents, once implanted, direct tissue contact with the one or more cells.

25. A method as claimed in claim 24 wherein each encapsulation involves presenting the one or more cells and a suitable alginate solution into a source of compatible cations thereby to entrap the one or more cells in a cation-alginate gel.

26. A method as claimed in claim 25 wherein the cation alginate gel is calcium-alginate gel, the alginate used in the solution is sodium alginate, and the islet and sodium alginate solution is presented as a droplet into a bath of suitable cations.

27. A method as claimed in claim 26 wherein the second layer of the capsule will be of a positively charged polymer material.

28. A method as claimed in claim 27 wherein the positively charged polymer material is poly-L-ornithine.

29. A method as claimed in claim 28 wherein the coatings comprise the following:
i) an inner layer of alginate,
ii) polyornithine,
iii) an outer layer of alginate.

30. A method as claimed in claim 29 wherein step iii) includes substantially uniform distribution of the one or more proteases in the one or more coatings (by mixing with the outer coating before application for example).

31. A method as claimed in claim 29 wherein step iii) includes distributing the one or more proteases throughout the outer coating in clusters (by mixing the clusters with the outer coating before application for example).

32. A method as claimed in claim 30 or 31 wherein the one or more proteases have the characteristics of proteases secreted by migrating parasitic nematodes.

33. A method as claimed in claim 32 wherein the nematodes are Ascaris suum, or stercoralis.

34. A method as claimed in claim 33 wherein the one or more proteases are neutral proteinases.

35. A method as claimed in claim 34 wherein the one or more proteases may be collagenases or proteoglycanases.

36. An intravenous preparation for administration to a patient suffering from cystic fibrosis comprising or including:
(a) a biological device as claimed in claim 1, and
(b) a pharmaceutically acceptable intravenous carrier.

37. A preparation as claimed in claim 36 wherein the intravenous preparation may be stored at a range of temperatures not less that 2 C. and not exceeding 30 C. without destabilisation and/or decomposition.

38. A method of treating a patient suffering from cystic fibrosis comprising or including the step of:
Intravenous administration to the patient of an intravenous preparation comprising
or including:
(a) a pharmaceutically acceptable intravenous carrier, and
(b) a biological device, wherein the biological device is capable, upon impaction in a lung capillary of a patient suffering from cystic fibrosis, of treating the patient.

39. A method as claimed in claim 38 wherein the biological device comprises or includes:
i) one or more cells exhibiting substantially normal CFTR production;
ii) one or more coatings of a suitable biocompatible material around the one of more cells;
iii) one or more proteases associated with the one or more coatings;
and wherein the size of the biological device is such that, upon introduction to the venous system of the patient the device will impact substantially in the region of a lung capillary of the patient, and wherein, upon impaction, by disruption of the outer surface of the device or otherwise, the one or more proteases are able to act upon a wall of the lung capillary, thereby allowing access (directly or indirectly) of the one or more cells to the lung epithelia.

40. A method as claimed in claim 39 wherein the diameter of the biological device is substantially within the range 20-80 micrometers.

41. A method as claimed in claim 40 wherein the one or more proteases are distributed substantially uniformly in the one or more coatings, whether it is uniformly though all of the one or more coatings or uniformly within one or more of the one or more coatings.

42. A method as claimed in claim 40 wherein the one or more proteases are distributed throughout the one or more coatings in clusters.

43. A method as claimed in claim 41 or 42 wherein the one or more proteases have the characteristics of proteases secreted by migrating parasitic nematodes.

44. A method as claimed in claim 43 wherein the nematodes are Ascaris suum, or stercoralis.

45. A method as claimed in claim 44 wherein the one or more proteases are neutral proteinases.

46. A method as claimed in claim 45 wherein the one or more proteases may be collagenases or proteo glycanases.

47. A method as claimed in claim 46 wherein the cells may include human lung cells and/or porcine lung cells and/or human lung stem cells.

48. A method as claimed in claim 47 wherein the one or more coatings around the cells provide a protective coating to the cells and are permeable to nutrients, including one or more of water, salts, glucose and amino acids.

49. A method as claimed in claim 48 wherein the one or more coatings are, or include alginate.

50. A method as claimed in claim 49 wherein the coatings comprise the following:
i) an inner layer of alginate,
ii) polyornithine,
iii) an outer layer of alginate.

51. A method as claimed in claim 50 wherein the proteases are in clusters, contained within a microcapsule, and are held in or on the outer layer of alginate.

52. A method as claimed in claim 51 wherein the microcapsule gelatin.

53. A method as claimed in claim 52 wherein the one or more cells have been obtained according to a process of isolation from a donor.

54. A method as claimed in claim 53 wherein the process of isolation includes exposure of the one or more cells to a medium containing one or more of:
Nicotinamide,
Liberase/Collagenase,
Lignocaine.

55. A method as claimed in claim 54 wherein the patient prior to and/or during and/or after administration of the intravenous preparation is treated with an oral dose of nicotinamide.

56. A biological device capable upon impaction in a lung capillary of a patient suffering from a condition or illness (“the condition”) of the lung or lung region, of treating the condition, comprising or including:
iii) one or more treatment species capable of treating the condition,
iv) One or more proteases associated with the one ore more treatment species,
wherein the size of the treatment species is such that, upon introduction to the venous system of the patient, the device will impact substantially in a region of a lung capillary of the patient, and wherein, upon impaction by disruption of the outer surface of the device or otherwise, the one or more proteases are able to act upon a wall of the lung capillary, thereby allowing access (directly or indirectly) of the one or more treatment species to the lung epithelia.

57. A biological device as claimed in claim 56 wherein the device includes one ore more external coatings of a biocompatible material around the one or more treatment species, the one or more proteases being distributed within the one ore more external coatings, and the diameter of the device is substantially within the range 20-80 micrometers.

58. A device as claimed in claim 57 wherein the one or more proteases have the characteristics of proteases secreted by the Ascaris roundworm, and are neutral proteinases.

59. A method of preparing a biological device capable, upon impaction in a lung capillary of a patient suffering from a condition or illness (“the condition”) of the lung or lung region of treating the condition comprising or including the steps of:
i) isolation of one or more treatment species capable of treating the condition; a step of coating the one or more cells with one or more coatings of a suitable biocompatible material;
ii) attaching or otherwise associating one or more proteases with the one or more coatings;
wherein the size of the biological device is such that, upon introduction to the venous system of the patient the device will impact substantially in the region of a lung capillary of the patient.

60. A method as claimed in claim 59 wherein the diameter of the biological device is substantially in the range 20-80 micrometers.

61. An intravenous preparation for administration to a patient suffering from a condition or illness of the lung or lung region comprising or including:
(c) a biological device as claimed in any one of claims 56 to 58, and
(d) a pharmaceutically acceptable intravenous carrier.

62. A method of treating a patient suffering from a condition or illness (“the condition”) of the lung or lung region comprising or including the step of:
Intravenous administration to the patient of an intravenous preparation comprising or including:
(c) a pharmaceutically acceptable intravenous carrier, and
(d) a biological device,
wherein the biological device is capable, upon impaction in a lung capillary of a patient suffering from the condition, of treating the patient.

63. A method as claimed in claim 62 wherein the diameter of the biological device is substantially within the range 20-80 micrometers.

64. A method as claimed in claim 63 wherein the biological device is as claimed in any one of claims 56 to 58.
